(19) United States
(12) Patent Application Publication
(10) Pub. No.: US 2025/0197314 A1
(43) Pub. Date: Jun. 19, 2025

(54) HYDROCARBON PRODUCTION SYSTEM AND HYDROCARBON PRODUCTION METHOD

(71) Applicant: IHI Corporation, Tokyo (JP)

(72) Inventors: Jun TSUJIKAWA, Tokyo (JP); Hiroyuki KAMATA, Tokyo (JP); Kentaro NARIAI, Tokyo (JP)

(73) Assignee: IHI Corporation, Tokyo (JP)

(21) Appl. No.: 19/068,393

(22) Filed: Mar. 3, 2025

Related U.S. Application Data
(63) Continuation of application No. PCT/JP2023/014236, filed on Apr. 6, 2023.

(30) Foreign Application Priority Data
Sep. 14, 2022 (JP) 2022-145886

(51) Int. Cl. C07C 1/12; B01D 53/02; B01D 53/86; B01J 8/00; C25B 15/08

(52) U.S. Cl. CPC C07C 1/12; B01D 53/02; B01D 53/8603; B01D 53/8671; B01D 53/869; B01J 8/00; C25B 15/081; B01D 2256/22; B01D 2257/104; B01D 2257/30; B01D 2258/0283

(57) ABSTRACT

A hydrocarbon production system includes: an impurity removal device that removes an impurity including any one or both of oxygen and a sulfur component from a mixed gas containing the impurity and carbon dioxide; a hydrocarbon production device, which includes a hydrocarbon synthesis catalyst for promoting a reaction for synthesizing hydrocarbon from carbon dioxide and hydrogen and synthesizes the hydrocarbon from the carbon dioxide contained in the mixed gas having the impurity removed by the impurity removal device and hydrogen; and a heat supply unit that supplies reaction heat generated in the hydrocarbon production device to the impurity removal device.

### CROSS REFERENCE TO RELATED APPLICATIONS

This application is a continuation application of International Application No. PCT/JP2023/014236, filed on Apr. 6, 2023, which claims priority to Japanese Patent Application No. 2022-145886 filed on Sep. 14, 2022, the entire contents of which are incorporated herein by reference.

### BACKGROUND ART

### Technical Field

The present disclosure relates to a hydrocarbon production system, and a hydrocarbon production method.

In a plant such as a thermal power plant, ironworks, or a boiler, a fossil fuel such as coal, heavy oil, or extra heavy oil is combusted. Thus, an exhaust gas containing carbon dioxide, which is generated as a result of the combustion of the fossil fuel, is emitted from the plant. Carbon dioxide is considered as a factor of global warming, and hence the suppression of emission of carbon dioxide into the atmosphere has been widely demanded.

Thus, there has been developed a technology for extracting carbon dioxide from the exhaust gas or the atmosphere and synthesizing hydrocarbon from the extracted carbon dioxide (for example, Patent Literature 1).

### CITATION LIST

### Patent Literature

Patent Literature 1: JP 2020-37535 A

### SUMMARY

### Technical Problem

In the above-mentioned technology for synthesizing hydrocarbon from carbon dioxide contained in the exhaust gas or the atmosphere, it has been desired that a technology for enabling the efficient use of thermal energy be developed.

Accordingly, the present disclosure has an object to provide a hydrocarbon production system and a hydrocarbon production method that enable the efficient use of thermal energy.

### Solution to Problem

In order to achieve the above-mentioned object, according to one aspect of the present disclosure, there is provided a hydrocarbon production system, including: an impurity removal device that removes an impurity including any one or both of oxygen and a sulfur component from a mixed gas containing the impurity and carbon dioxide; a hydrocarbon production device, which includes a hydrocarbon synthesis catalyst for promoting a reaction for synthesizing hydrocarbon from carbon dioxide and hydrogen and synthesizes the hydrocarbon from the carbon dioxide contained in the mixed gas having the impurity removed by the impurity removal device and hydrogen; and a heat supply unit that supplies reaction heat generated in the hydrocarbon production device to the impurity removal device.

The above-mentioned hydrocarbon production system may further include a wet desulfurization device that removes the sulfur component from the mixed gas. The impurity removal device may include a catalytic desulfurization device, which includes a desulfurization catalyst and is to be supplied with the mixed gas that has been treated by the wet desulfurization device, and the heat supply unit may supply the reaction heat to the catalytic desulfurization device.

The above-mentioned hydrocarbon production system may further include a wet desulfurization device that removes the sulfur component from the mixed gas. The impurity removal device may include: an oxygen removal device, which includes an oxygen removal catalyst and is to be supplied with the mixed gas that has been treated by the wet desulfurization device and hydrogen; and a catalytic desulfurization device, which includes a desulfurization catalyst and is to be supplied with the mixed gas that has been treated by the oxygen removal device, and the heat supply unit may supply the reaction heat to any one or both of the catalytic desulfurization device and the oxygen removal device.

The above-mentioned hydrocarbon production system may further include a wet desulfurization device that removes the sulfur component from the mixed gas. The impurity removal device may include: a dry desulfurization device, which includes an adsorbent for adsorbing the sulfur component and is to be supplied with the mixed gas that has been treated by the wet desulfurization device; an oxygen removal device, which includes an oxygen removal catalyst and is to be supplied with the mixed gas that has been treated by the dry desulfurization device and hydrogen; and a catalytic desulfurization device, which includes a desulfurization catalyst and is to be supplied with the mixed gas that has been treated by the oxygen removal device, and the heat supply unit may supply the reaction heat to any one or more of the catalytic desulfurization device, the oxygen removal device, and the dry desulfurization device.

The above-mentioned hydrocarbon production system may further include a wet desulfurization device that removes the sulfur component from the mixed gas. The impurity removal device may include an oxygen removal device, which includes an oxygen removal catalyst and is to be supplied with the mixed gas that has been treated by the wet desulfurization device and hydrogen, and the heat supply unit may supply the reaction heat to the oxygen removal device.

The above-mentioned hydrocarbon production system may further include a wet desulfurization device that removes the sulfur component from the mixed gas. The impurity removal device may include: a dry desulfurization device, which includes an adsorbent for adsorbing the sulfur component and is to be supplied with the mixed gas that has been treated by the wet desulfurization device; and an oxygen removal device, which includes an oxygen removal catalyst and is to be supplied with the mixed gas that has been treated by the dry desulfurization device and hydrogen, and the heat supply unit may supply the reaction heat to any one or both of the oxygen removal device and the dry desulfurization device.

The above-mentioned impurity removal device may include an oxygen removal device, which includes an oxygen removal catalyst and is to be supplied with hydrogen, and the heat supply unit may supply the reaction heat to the oxygen removal device.

The above-mentioned heat supply unit may include a heat medium for recovering the reaction heat generated in the hydrocarbon production device, and the heat medium may heat the catalytic desulfurization device and then heat the oxygen removal device.

The above-mentioned hydrocarbon production system may further include a heat exchanger that allows heat exchange between the mixed gas that has been treated by the wet desulfurization device and the mixed gas that has been treated by the impurity removal device.

The above-mentioned hydrocarbon production system may further include: a water electrolyzer that electrolyzes water to produce hydrogen and oxygen; and an activation device that supplies, when the hydrocarbon production device is to be activated, the oxygen produced by the water electrolyzer. The hydrogen produced by the water electrolyzer may be supplied to the oxygen removal catalyst and the hydrocarbon production device.

In order to achieve the above-mentioned object, according to one aspect of the present disclosure, there is provided a hydrocarbon production method, including: removing an impurity including any one or both of oxygen and a sulfur component from a mixed gas containing the impurity and carbon dioxide in an impurity removal device; causing a reaction between the mixed gas having the impurity removed and hydrogen; and supplying reaction heat generated in the reaction between carbon dioxide contained in the mixed gas having the impurity removed and the hydrogen to the impurity removal device.

### Effects

According to the present disclosure, the efficient use of thermal energy is enabled.

### BRIEF DESCRIPTION OF DRAWINGS

FIG. 1 is a diagram for illustrating a hydrocarbon production system according to a first embodiment.

FIG. 2 is a flowchart for illustrating a flow of processing of a hydrocarbon production method according to the first embodiment.

FIG. 3 is a diagram for illustrating a hydrocarbon production system according to a second embodiment.

FIG. 4 is a diagram for illustrating a hydrocarbon production system according to a third embodiment.

FIG. 5 is a diagram for illustrating a hydrocarbon production system according to a fourth embodiment.

FIG. 6 is a diagram for illustrating a hydrocarbon production system according to a fifth embodiment.

FIG. 7 is a diagram for illustrating a hydrocarbon production system according to a sixth embodiment.

### DESCRIPTION OF EMBODIMENT

Now, with reference to the attached drawings, embodiments of the present disclosure are described in detail. The dimensions, materials, and other specific numerical values represented in the embodiments are merely examples used for facilitating the understanding of the disclosure, and do not limit the present disclosure otherwise particularly noted. Elements having substantially the same functions and configurations herein and in the drawings are denoted by the same reference symbols to omit redundant description thereof. Further, illustration of elements with no direct relationship to the present disclosure is omitted.

### First Embodiment: Hydrocarbon Production System

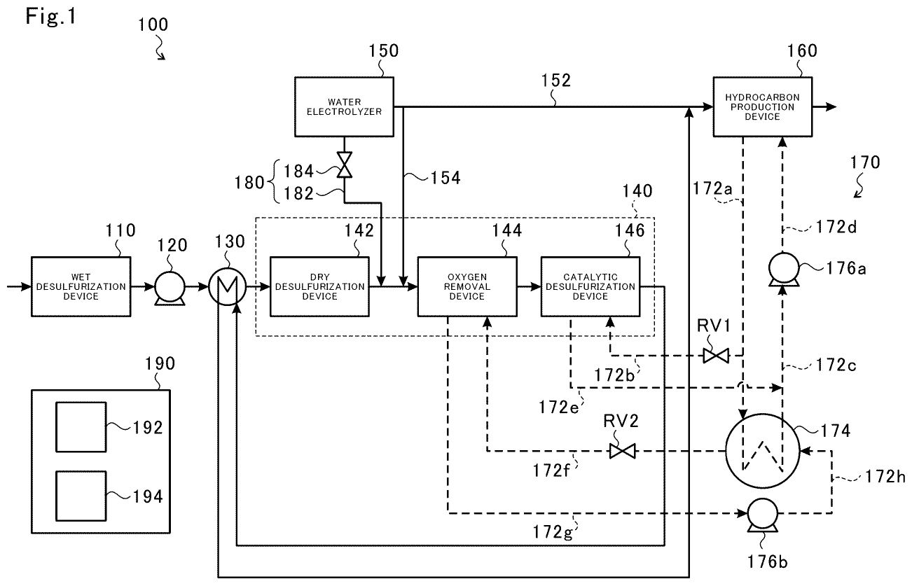

FIG. 1 is a diagram for illustrating a hydrocarbon production system 100 according to a first embodiment. As illustrated in FIG. 1, the hydrocarbon production system 100 includes a wet desulfurization device 110, a compressor 120, a heat exchanger 130, an impurity removal device 140, a water electrolyzer 150, a hydrocarbon production device 160, a heat supply unit 170, an activation device 180, and a central controller 190. In FIG. 1, the solid-line arrows indicate the flows of gases such as a mixed gas, carbon dioxide (CO₂), oxygen (O₂), hydrogen (H₂), and hydrocarbons (CH₄, (CH₂)ₙ). Further, in FIG. 1, the broken-line arrows indicate the flows of heat media.

The hydrocarbon production system 100 extracts carbon dioxide from a mixed gas and causes a reaction between the extracted carbon dioxide and hydrogen to thereby produce hydrocarbon. In this embodiment, the mixed gas contains a sulfur component, oxygen, and carbon dioxide. The mixed gas is, for example, a gas generated by combustion of a fossil fuel with oxygen.

The wet desulfurization device 110 removes a sulfur component from the mixed gas. The wet desulfurization device 110 is, for example, a spray tower that sprays a liquid containing a basic substance into the mixed gas. The basic substance is, for example, sodium hydroxide or magnesium hydroxide. The wet desulfurization device 110 decreases a concentration of the sulfur component in the mixed gas to, for example, about 10 ppm. The wet desulfurization device 110 is operated at, for example, normal temperature or higher and 60° C. or lower. The normal temperature is, for example, 25° C.

A suction side of the compressor 120 is connected to the wet desulfurization device 110. A discharge side of the compressor 120 is connected to the heat exchanger 130 described later. The compressor 120 boosts a pressure of the mixed gas that has been treated by the wet desulfurization device 110 and then supplies the boosted mixed gas to the heat exchanger 130. The compressor 120 is, for example, a pump or a blower.

The heat exchanger 130 allows heat exchange between the mixed gas that has been treated by the wet desulfurization device 110 and has been discharged by the compressor 120 and a mixed gas that has been treated by a catalytic desulfurization device 146 described later.

The impurity removal device 140 removes impurities from the mixed gas. In this embodiment, the impurities are a sulfur component and oxygen. Further, in this embodiment, the impurity removal device 140 includes a dry desulfurization device 142, an oxygen removal device 144, and the catalytic desulfurization device 146.

The mixed gas that has passed through the wet desulfurization device 110, the compressor 120, and the heat exchanger 130 is supplied to the dry desulfurization device 142. The dry desulfurization device 142 further removes the sulfur component from the mixed gas that has been treated by the wet desulfurization device 110. The dry desulfurization device 142 removes the sulfur component from the mixed gas in a gas phase. In this embodiment, the dry desulfurization device 142 includes an adsorbent for adsorbing a sulfur component. The dry desulfurization device 142 is operated at normal temperature or higher and 60° C. or lower. The adsorbent is, for example, an alumina-based adsorbent or activated carbon.

The mixed gas that has been treated by the dry desulfurization device 142 and hydrogen that has been produced by the water electrolyzer 150 described later are supplied to the oxygen removal device 144. The oxygen removal device 144 causes a reaction between oxygen contained in the mixed gas and hydrogen to thereby produce water. As a result, oxygen is removed from the mixed gas in the oxygen removal device 144. The oxygen removal device 144 removes oxygen from the mixed gas in a gas phase. In this embodiment, the oxygen removal device 144 includes an oxygen removal catalyst. The oxygen removal device 144 is operated at normal temperature or higher and 200° C. or lower, for example, 50° C. or higher and 200° C. or lower. The reaction between oxygen and hydrogen that progresses in the oxygen removal device 144 is an exothermic reaction.

In this embodiment, examples of the oxygen removal catalyst include a platinum (Pt)-based catalyst, a palladium (Pd)-based catalyst, and a nickel (Ni)-based catalyst. The oxygen removal catalyst has, for example, a pellet shape or a honeycomb shape.

The mixed gas that has been treated by the oxygen removal device 144 is supplied to the catalytic desulfurization device 146. The catalytic desulfurization device 146 further removes the sulfur component from the mixed gas that has been treated by the oxygen removal device 144. The catalytic desulfurization device 146 removes the sulfur component from the mixed gas in a gas phase. In this embodiment, the catalytic desulfurization device 146 includes a desulfurization catalyst. The catalytic desulfurization device 146 is operated at 200° C. or higher and 300° C. or lower.

In this embodiment, the desulfurization catalyst in the catalytic desulfurization device 146 is a catalyst that performs desulfurization under a hydrogen atmosphere, that is, under a reducing atmosphere. The desulfurization catalyst in the catalytic desulfurization device 146 is a zinc (Zn)-based catalyst, a nickel-based catalyst, or a cobalt (Co)-based catalyst. The desulfurization catalyst in the catalytic desulfurization device 146 has, for example, a pellet shape or a honeycomb shape.

The water electrolyzer 150 electrolyzes water to thereby produce hydrogen and oxygen. The water electrolyzer 150 electrolyzes water with use of, for example, electric power generated through renewable energy.

The hydrogen produced by the water electrolyzer 150 is supplied to the hydrocarbon production device 160 and the oxygen removal device 144 via flow paths 152 and 154. The flow path 152 connects a hydrogen output port of the water electrolyzer 150 and the hydrocarbon production device 160 to each other. The flow path 154 connects the hydrogen output port of the water electrolyzer 150 and the oxygen removal device 144 to each other.

The mixed gas that has been treated by the impurity removal device 140 (catalytic desulfurization device 146) and the hydrogen produced by the water electrolyzer 150 are supplied to the hydrocarbon production device 160. The hydrocarbon production device 160 synthesizes hydrocarbon from carbon dioxide contained in the mixed gas having the sulfur component and oxygen removed by the impurity removal device 140 and the hydrogen produced by the water electrolyzer 150. The hydrocarbon production device 160 causes a reaction between carbon dioxide and hydrogen in a gas phase. In this embodiment, the hydrocarbon production device 160 includes a hydrocarbon synthesis catalyst. The hydrocarbon synthesis catalyst is a catalyst that promotes a reaction for synthesizing hydrocarbon from carbon dioxide and hydrogen. The hydrocarbon production device 160 is operated at 300° C. or higher and 350° C. or lower.

In this embodiment, the hydrocarbon synthesis catalyst is a catalyst that promotes a methanation reaction expressed by Formula (1) or a catalyst that promotes a Fischer-Tropsch (FT) synthesis reaction expressed by Formula (2). The catalyst that promotes the methanation reaction is, for example, a nickel-based catalyst. The catalyst that promotes the FT synthesis reaction is, for example, an iron (Fe)-based catalyst.

CO₂+4H₂→CH₄+2H₂O . . . Formula (1)

nCO₂+3nH₂→(CH₂)ₙ+2nH₂O . . . Formula (2)

In Formula (2), “n” is, for example, 2 or more and 4 or less. The methanation reaction and the FT synthesis reaction are each an exothermic reaction.

The hydrocarbon synthesis catalyst has, for example, a pellet shape or a honeycomb shape.

The heat supply unit 170 supplies reaction heat generated in the hydrocarbon production device 160 to the impurity removal device 140. In this embodiment, the heat supply unit 170 supplies the reaction heat generated in the hydrocarbon production device 160 to the catalytic desulfurization device 146 and the oxygen removal device 144.

In this embodiment, the heat supply unit 170 includes flow paths 172a to 172h, a heat exchanger 174, and feeding devices 176a and 176b. The flow paths 172a to 172e are flow paths through which a first heat medium circulates. The flow paths 172f to 172h are flow paths through which a second heat medium circulates.

The flow path 172a connects the hydrocarbon production device 160 and the heat exchanger 174 to each other. The flow path 172b connects the flow path 172a and the catalytic desulfurization device 146 to each other. The first heat medium, which has been heated through recovery of the reaction heat generated in the hydrocarbon production device 160, is supplied to the heat exchanger 174 through the flow path 172a and is supplied to the catalytic desulfurization device 146 through the flow paths 172a and 172b.

The flow path 172c connects the heat exchanger 174 and a suction side of the feeding device 176a to each other. The flow path 172d connects a discharge side of the feeding device 176a and the hydrocarbon production device 160 to each other. The flow path 172e connects the catalytic desulfurization device 146 and the flow path 172c to each other.

The feeding device 176a is, for example, a pump. When the feeding device 176a is operated, the first heat medium circulates through the hydrocarbon production device 160, the catalytic desulfurization device 146, and the heat exchanger 174 via the flow paths 172a to 172e.

The flow path 172f connects the heat exchanger 174 and the oxygen removal device 144 to each other. The flow path 172g connects the oxygen removal device 144 and a suction side of the feeding device 176b to each other. The flow path 172h connects a discharge side of the feeding device 176b and the heat exchanger 174 to each other.

The feeding device 176b is, for example, a pump. When the feeding device 176b is operated, the second heat medium circulates through the heat exchanger 174 and the oxygen removal device 144 via the flow paths 172f to 172h.

The heat exchanger 174 allows heat exchange between the first heat medium and the second heat medium.

The reaction heat generated in the hydrocarbon production device 160 is first transferred to the first heat medium. Then, the heat of the first heat medium is supplied to the catalytic desulfurization device 146. Further, the heat of the first heat medium is transferred to the second heat medium via the heat exchanger 174, and is then supplied to the oxygen removal device 144.

Further, a flow rate control valve RV1 is provided to the flow path 172b. A flow rate control valve RV2 is provided to the flow path 172f. An opening degree of the flow rate control valve RV1 and an opening degree of the flow rate control valve RV2 are adjusted by a heat controller 192 described later.

When the hydrocarbon production device 160 is to be activated, the activation device 180 supplies the oxygen produced by the water electrolyzer 150 to the oxygen removal device 144. In this embodiment, the activation device 180 includes a flow path 182 and an on-off valve 184. The flow path 182 connects an oxygen output port of the water electrolyzer 150 and the oxygen removal device 144 to each other. The on-off valve 184 is provided to the flow path 182. The on-off valve 184 opens and closes the flow path 182. The on-off valve 184 is controlled to be opened and closed by an activation controller 194 described later.

The central controller 190 is formed of a semiconductor integrated circuit including a central processing unit (CPU). The central controller 190 reads out, for example, a program and a parameter each for operating the CPU from a ROM. The central controller 190 manages and controls the entire hydrocarbon production system 100 in cooperation with a RAM serving as a working area and other electronic circuit.

In this embodiment, the central controller 190 also functions as the heat controller 192 and the activation controller 194.

The heat controller 192 adjusts the opening degree of the flow rate control valve RV1 so that a temperature of the catalytic desulfurization device 146 is maintained at an active temperature of the desulfurization catalyst. In this embodiment, the heat controller 192 adjusts the opening degree of the flow rate control valve RV1 so that the temperature of the catalytic desulfurization device 146 becomes 200° C. or higher and 300° C. or lower.

Further, the heat controller 192 adjusts the opening degree of the flow rate control valve RV2 so that a temperature of the oxygen removal device 144 is maintained at an active temperature of the oxygen removal catalyst. In this embodiment, the heat controller 192 adjusts the opening degree of the flow rate control valve RV2 so that the temperature of the oxygen removal device 144 becomes 50° C. or higher and 200° C. or lower.

When the hydrocarbon production device 160 is to be activated, the activation controller 194 opens the on-off valve 184. Further, when the temperatures of the oxygen removal device 144, the catalytic desulfurization device 146, and the hydrocarbon production device 160 reach an operating temperature, the activation controller 194 closes the on-off valve 184.

### Hydrocarbon Production Method

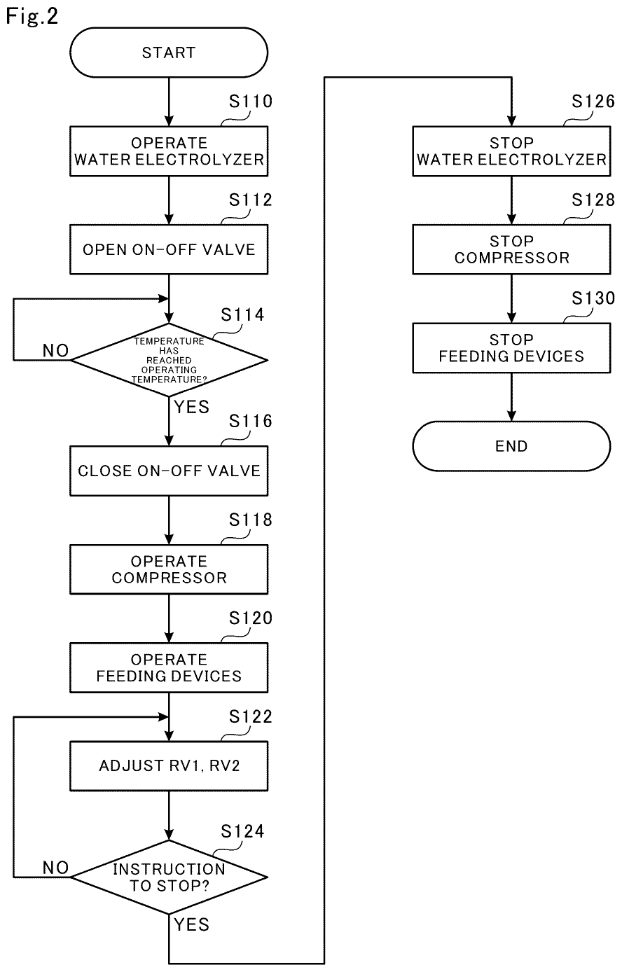

Subsequently, a hydrocarbon production method using the above-mentioned hydrocarbon production system 100 is described. FIG. 2 is a flowchart for illustrating a flow of processing of the hydrocarbon production method according to this embodiment.

As illustrated in FIG. 2, the hydrocarbon production method includes a water electrolyzer operating process S110, an opening process S112, a temperature determination process S114, a closing process S116, a compressor operating process S118, a feeding device operating process S120, an adjustment process S122, a stop determination process S124, a water electrolyzer stop process S126, a compressor stop process S128, and a feeding device stop process S130. Now, each of the processes is described.

### Water Electrolyzer Operating Process S110

The activation controller 194 starts the operation of the water electrolyzer 150. As a result, water is electrolyzed by the water electrolyzer 150 to produce hydrogen and oxygen. The hydrogen produced by the water electrolyzer 150 is supplied to the oxygen removal device 144 and the hydrocarbon production device 160.

### Opening Process S112

The activation controller 194 opens the on-off valve 184. As a result, the oxygen produced by the water electrolyzer 150 is supplied to the oxygen removal device 144. Thus, an exothermic reaction between the water and the oxygen, which have been produced by the water electrolyzer 150, progresses in the oxygen removal device 144.

### Temperature Determination Process S114

The activation controller 194 determines whether or not the temperature of the oxygen removal device 144 has reached the operating temperature. The operating temperature is the active temperature of the oxygen removal catalyst in the oxygen removal device 144. As a result, when it is determined that the temperature of the oxygen removal device 144 is less than the operating temperature (NO in Step S114), the activation controller 194 repeats the temperature determination process S114. Meanwhile, when it is determined that the temperature of the oxygen removal device 144 has reached the operating temperature (YES in Step S114), the activation controller 194 advances the processing to the closing process S116.

### Closing Process S116

The activation controller 194 closes the on-off valve 184. As a result, the supply of oxygen produced by the water electrolyzer 150 to the oxygen removal device 144 is stopped.

### Compressor Operating Process S118

The activation controller 194 starts the operations of the wet desulfurization device 110 and the compressor 120. Further, as a result, the mixed gas is supplied to the wet desulfurization device 110 and is desulfurized by the wet desulfurization device 110. Further, the mixed gas, which has been desulfurized by the wet desulfurization device 110, is supplied to the impurity removal device 140 (the dry desulfurization device 142, the oxygen removal device 144, and the catalytic desulfurization device 146), and impurities (a sulfur component and oxygen) are removed by the impurity removal device 140. The mixed gas having the impurities removed in this manner is supplied to the hydrocarbon production device 160. Then, a reaction between carbon dioxide contained in the mixed gas and the hydrogen supplied from the water electrolyzer 150 is caused in the hydrocarbon production device 160 to thereby produce hydrocarbon.

### Feeding Device Operating Process S120

The heat controller 192 starts the operation of the feeding device 176a. Then, the first heat medium circulates through the hydrocarbon production device 160, the catalytic desulfurization device 146, and the heat exchanger 174. As a result, the reaction heat generated in the hydrocarbon production device 160 is supplied to the catalytic desulfurization device 146 via the first heat medium.

Further, the heat controller 192 starts the operation of the feeding device 176b. Then, the second heat medium circulates through the heat exchanger 174 and the oxygen removal device 144. As a result, the reaction heat generated in the hydrocarbon production device 160 is supplied to the oxygen removal device 144 via the first heat medium, the heat exchanger 174, and the second heat medium.

### Adjustment Process S122

The heat controller 192 adjusts the opening degree of the flow rate control valve RV1 so that the temperature of the catalytic desulfurization device 146 is maintained at the active temperature of the desulfurization catalyst. Further, the heat controller 192 adjusts the opening degree of the flow rate control valve RV2 so that the temperature of the oxygen removal device 144 is maintained at the active temperature of the oxygen removal catalyst.

During the operation of the hydrocarbon production device 160, the adjustment process S122 is continuously carried out.

### Stop Determination Process S124

The central controller 190 determines whether or not an instruction to stop the operation has been received from a user. As a result, when it is determined that the instruction to stop the operation has not been received (NO in Step S124), the central controller 190 returns the processing to the adjustment process S122. Meanwhile, when it is determined that the instruction to stop the operation has been received (YES in Step S124), the central controller 190 advances the processing to the water electrolyzer stop process S126.

### Water electrolyzer Stop Process S126

The central controller 190 stops the operation of the water electrolyzer 150.

### Compressor Stop Process S128

The central controller 190 stops the operations of the wet desulfurization device 110 and the compressor 120.

### Feeding Device Stop Process S130

The central controller 190 stops the operations of the feeding devices 176a and 176b.

As described above, the hydrocarbon production system 100 according to this embodiment includes the wet desulfurization device 110, the dry desulfurization device 142, and the catalytic desulfurization device 146. As a result, the hydrocarbon production system 100 can decrease the concentration of the sulfur component contained in the mixed gas supplied to the hydrocarbon production device 160 to be extremely low. Accordingly, the hydrocarbon production system 100 can reduce a poisoning amount of the hydrocarbon synthesis catalyst of the hydrocarbon production device 160. Hence, the hydrocarbon production system 100 can reduce the amount of hydrocarbon synthesis catalyst in the hydrocarbon production device 160. Further, the hydrocarbon production system 100 can reduce a frequency of replacement of the poisoned hydrocarbon synthesis catalyst with a new unpoisoned hydrocarbon synthesis catalyst in the hydrocarbon production device 160. Accordingly, the hydrocarbon production system 100 can reduce running cost of the hydrocarbon production device 160.

Further, as described above, the hydrocarbon production system 100 includes the dry desulfurization device 142 on an upstream side of the oxygen removal device 144. Thus, even when the oxygen removal catalyst of the oxygen removal device 144 has low tolerance to sulfur, the dry desulfurization device 142 can suppress the deterioration of an oxygen removal function.

Further, as described above, the hydrocarbon production system 100 includes the oxygen removal device 144. Thus, the oxygen removal device 144 can suppress undesirable mixture of oxygen into the catalytic desulfurization device 146 that performs desulfurization under a reducing atmosphere. Accordingly, the oxygen removal device 144 can suppress the deterioration of a desulfurization function of the catalytic desulfurization device 146.

Further, the oxygen removal device 144 can suppress undesirable mixture of oxygen into the hydrocarbon production device 160. As a result, the oxygen removal device 144 can suppress a reduction in reaction efficiency in the hydrocarbon production device 160.

Further, as described above, the hydrocarbon production system 100 and the hydrocarbon production method using this system allow the reaction heat generated in the hydrocarbon production device 160 to be supplied to the catalytic desulfurization device 146 and the oxygen removal device 144. Thus, the hydrocarbon production system 100 and the hydrocarbon production method using this system allow the reaction heat to be used for thermal energy for maintaining the desulfurization catalyst of the catalytic desulfurization device 146 and the oxygen removal catalyst of the oxygen removal device 144 at their active temperatures. Specifically, the hydrocarbon production system 100 enables the efficient use of thermal energy. Hence, the hydrocarbon production system 100 and the hydrocarbon production method using this system can reduce cost required for the thermal energy for maintaining the desulfurization catalyst of the catalytic desulfurization device 146 and the oxygen removal catalyst of the oxygen removal device 144 at their active temperatures. Accordingly, the hydrocarbon production system 100 and the hydrocarbon production method using this system can reduce running cost for the impurity removal device 140.

Further, as described above, the active temperature of the desulfurization catalyst of the catalytic desulfurization device 146 is higher than the active temperature of the oxygen removal catalyst of the oxygen removal device 144. After heating the catalytic desulfurization device 146 with the first heat medium, the heat supply unit 170 heats the oxygen removal device 144 with the first heat medium and the second heat medium. As a result, the heat supply unit 170 can efficiently heat both of the catalytic desulfurization device 146 and the oxygen removal device 144.

Further, as described above, the hydrocarbon production system 100 include the heat exchanger 130. As a result, the heat exchanger 130 can heat the mixed gas before being supplied to the dry desulfurization device 142. Accordingly, the heat exchanger 130 can prevent the occurrence of dew condensation in the dry desulfurization device 142. Hence, the heat exchanger 130 can suppress the deterioration of a desulfurization function of the dry desulfurization device 142.

Further, as described above, the hydrocarbon production system 100 includes the activation device 180. As a result, when the hydrocarbon production device 160 is to be activated, the activation device 180 enables the exothermic reaction between hydrogen and oxygen to progress in the oxygen removal device 144. Thus, when the hydrocarbon production device 160 is to be activated, the activation device 180 can warm the oxygen removal device 144, the catalytic desulfurization device 146, and the hydrocarbon production device 160 with the reaction heat generated in the oxygen removal device 144. Hence, the activation device 180 can reduce cost required for warming.

### Second Embodiment: Hydrocarbon Production System

In the first embodiment, there has been given, as an example, the case in which the hydrocarbon production system 100 includes the dry desulfurization device 142. However, when the oxygen removal catalyst has high tolerance to sulfur, the dry desulfurization device 142 may be omitted.

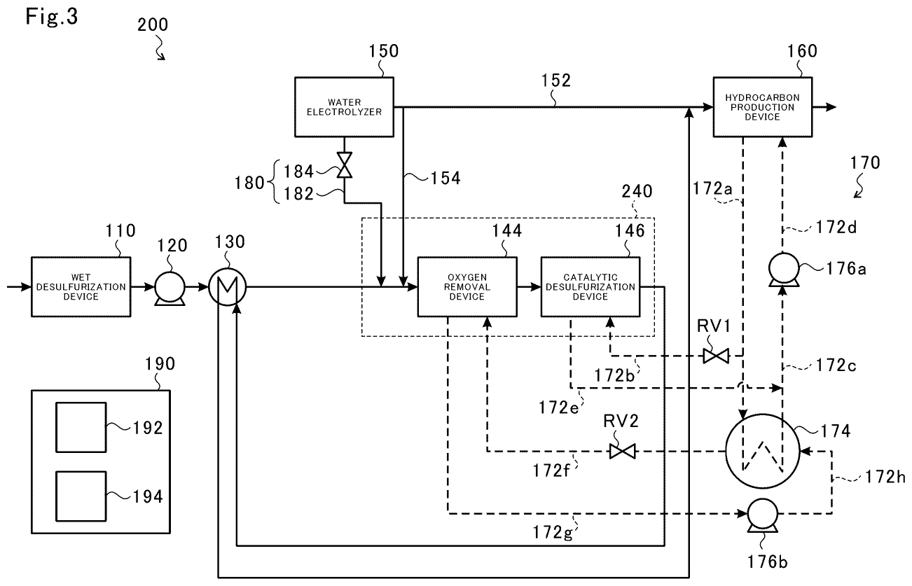

FIG. 3 is a diagram for illustrating a hydrocarbon production system 200 according to a second embodiment. As illustrated in FIG. 3, the hydrocarbon production system 200 includes a wet desulfurization device 110, a compressor 120, a heat exchanger 130, an impurity removal device 240, a water electrolyzer 150, a hydrocarbon production device 160, a heat supply unit 170, an activation device 180, and a central controller 190. In FIG. 3, the solid-line arrows indicate the flows of gases such as a mixed gas, carbon dioxide, oxygen, hydrogen, and hydrocarbon. Further, in FIG. 3, the broken-line arrows indicate the flows of heat media.

Substantially the same components as those in the above-mentioned hydrocarbon production system 100 are denoted by the same reference symbols, and description thereof is omitted.

The impurity removal device 240 includes an oxygen removal device 144 and a catalytic desulfurization device 146. Thus, in this embodiment, a mixed gas that has passed through the wet desulfurization device 110, the compressor 120, and the heat exchanger 130 is supplied to the oxygen removal device 144.

Further, in this embodiment, an oxygen removal catalyst of the oxygen removal device 144 has higher tolerance to sulfur than that of the oxygen removal catalyst of the oxygen removal device 144 of the hydrocarbon production system 100. The oxygen removal catalyst of the oxygen removal device 144 according to this embodiment is, for example, a platinum-based catalyst, a palladium-based catalyst, or a nickel-based catalyst. The oxygen removal catalyst has, for example, a pellet shape or a honeycomb shape.

As described above, as compared to the hydrocarbon production system 100, the hydrocarbon production system 200 according to this embodiment allows the omission of the dry desulfurization device 142. Thus, the hydrocarbon production system 200 enables the efficient use of thermal energy while reducing cost required for the dry desulfurization device 142.

### Third Embodiment: Hydrocarbon Production System

In the first embodiment, there has been given, as an example, the case in which the hydrocarbon production system 100 includes the catalytic desulfurization device 146. However, when a hydrocarbon synthesis catalyst has high tolerance to sulfur, the catalytic desulfurization device 146 may be omitted.

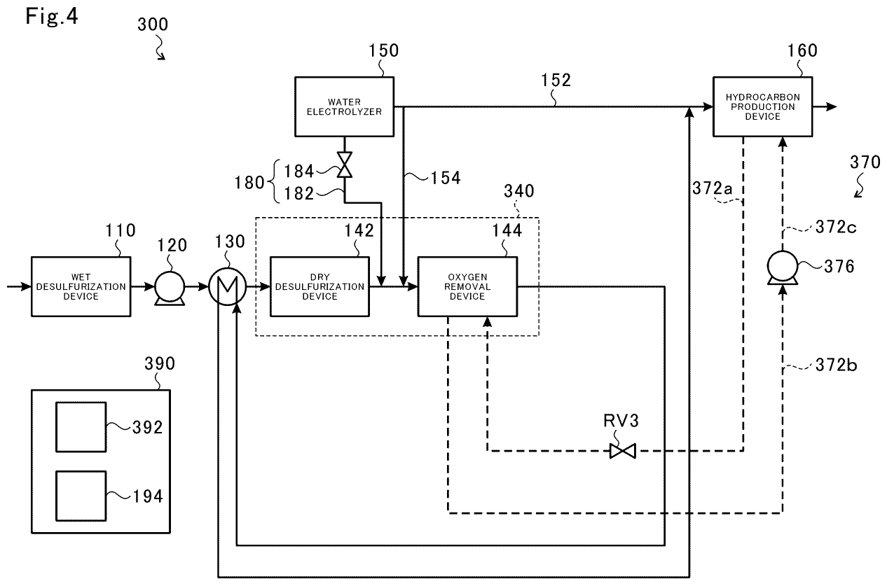

FIG. 4 is a diagram for illustrating a hydrocarbon production system 300 according to a third embodiment. As illustrated in FIG. 4, the hydrocarbon production system 300 includes a wet desulfurization device 110, a compressor 120, a heat exchanger 130, an impurity removal device 340, a water electrolyzer 150, a hydrocarbon production device 160, a heat supply unit 370, an activation device 180, and a central controller 390. In FIG. 4, the solid-line arrows indicate the flows of gases such as a mixed gas, carbon dioxide, oxygen, hydrogen, and hydrocarbon. Further, in FIG. 4, the broken-line arrows indicate the flows of heat media.

Substantially the same components as those in the above-mentioned hydrocarbon production system 100 are denoted by the same reference symbols, and description thereof is omitted.

The impurity removal device 340 includes a dry desulfurization device 142 and an oxygen removal device 144.

In this embodiment, the heat exchanger 130 allows heat exchange between a mixed gas that has been treated by the wet desulfurization device 110 and has been discharged from the compressor 120 and a mixed gas that has been treated by the oxygen removal device 144.

Further, in this embodiment, the mixed gas that has been treated by the oxygen removal device 144 and hydrogen produced by the water electrolyzer 150 are supplied to the hydrocarbon production device 160.

Further, in this embodiment, a hydrocarbon synthesis catalyst of the hydrocarbon production device 160 has higher tolerance to sulfur than that of the hydrocarbon synthesis catalyst of the hydrocarbon production device 160 of the hydrocarbon production system 100. The hydrocarbon synthesis catalyst of the hydrocarbon production device 160 according to this embodiment is, for example, a nickel-based catalyst or an iron-based catalyst. The hydrocarbon synthesis catalyst has, for example, a pellet shape or a honeycomb shape.

The heat supply unit 370 supplies reaction heat generated in the hydrocarbon production device 160 to the oxygen removal device 144. In this embodiment, the heat supply unit 370 includes flow paths 372a to 372c and a feeding device 376.

The flow path 372a connects the hydrocarbon production device 160 and the oxygen removal device 144 to each other. The flow path 372b connects the oxygen removal device 144 and a suction side of the feeding device 376 to each other. The flow path 372c connects a discharge side of the feeding device 376 and the hydrocarbon production device 160 to each other.

The feeding device 376 is, for example, a pump. When the feeding device 376 is operated, a heat medium circulates through the hydrocarbon production device 160 and the oxygen removal device 144 via the flow paths 372a to 372c. As a result, the reaction heat generated in the hydrocarbon production device 160 is supplied to the oxygen removal device 144.

Further, a flow rate control valve RV3 is provided to the flow path 372a. An opening degree of the flow rate control valve RV3 is adjusted by a heat controller 392 described later.

The central controller 390 is formed of a processing unit (CPU). The central controller 390 reads out, for example, a program and a parameter each for operating the CPU from a ROM. The central controller 390 manages and controls the entire hydrocarbon production system 300 in cooperation with a RAM serving as a working area and other electronic circuit.

In this embodiment, the central controller 390 also functions as the heat controller 392 and an activation controller 194.

The heat controller 392 adjusts the opening degree of the flow rate control valve RV3 so that a temperature of the oxygen removal device 144 is maintained at an activation temperature of an oxygen removal catalyst. In this embodiment, the heat controller 392 adjusts the opening degree of the flow rate control valve RV3 so that the temperature of the oxygen removal device 144 becomes 50° C. or higher and 200° C. or lower.

As described above, as compared to the hydrocarbon production system 100, the hydrocarbon production system 300 according to this embodiment allows the omission of the catalytic desulfurization device 146. Thus, the hydrocarbon production system 300 enables the efficient use of thermal energy while reducing cost required for the catalytic desulfurization device 146.

### Fourth Embodiment: Hydrocarbon Production System

In the first embodiment, there has been given, as an example, the case in which the hydrocarbon production system 100 includes the dry desulfurization device 142 and the catalytic desulfurization device 146. However, when the oxygen removal catalyst and the hydrocarbon synthesis catalyst have high tolerance to sulfur, the dry desulfurization device 142 and the catalytic desulfurization device 146 may be omitted.

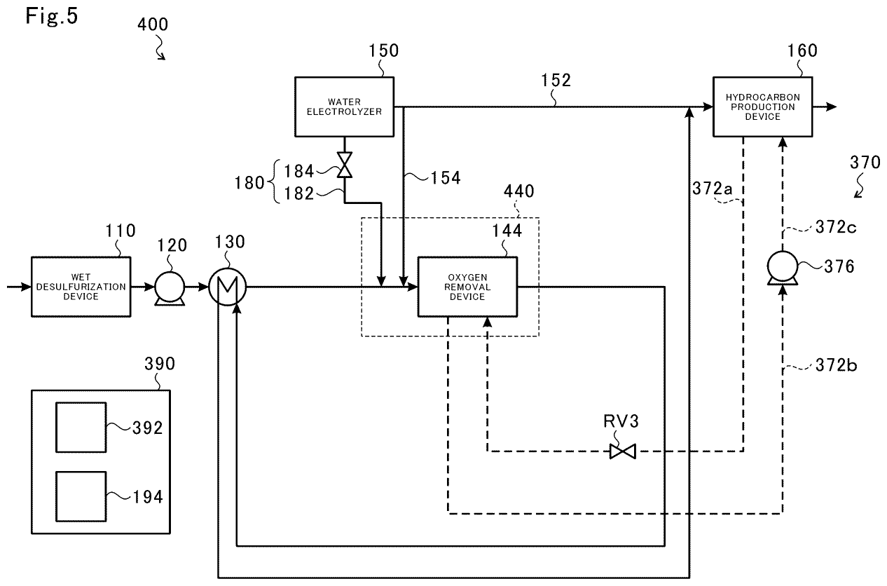

FIG. 5 is a diagram for illustrating a hydrocarbon production system 400 according to a fourth embodiment. As illustrated in FIG. 5, the hydrocarbon production system 400 includes a wet desulfurization device 110, a compressor 120, a heat exchanger 130, an impurity removal device 440, a water electrolyzer 150, a hydrocarbon production device 160, a heat supply unit 370, an activation device 180, and a central controller 390. In FIG. 5, the solid-line arrows indicate the flows of gases such as a mixed gas, carbon dioxide, oxygen, hydrogen, and hydrocarbon. Further, in FIG. 5, the broken-line arrows indicate the flows of heat media.

Substantially the same components as those in the above-mentioned hydrocarbon production system 300 are denoted by the same reference symbols, and description thereof is omitted.

The impurity removal device 440 includes an oxygen removal device 144.

In this embodiment, the heat exchanger 130 allows heat exchange between a mixed gas that has been treated by the wet desulfurization device 110 and has been discharged from the compressor 120 and a mixed gas that has been treated by the oxygen removal device 144.

Further, in this embodiment, the mixed gas that has been treated by the oxygen removal device 144 and hydrogen produced by the water electrolyzer 150 are supplied to the hydrocarbon production device 160.

Further, in this embodiment, an oxygen removal catalyst of the oxygen removal device 144 has higher tolerance to sulfur than that of the oxygen removal catalyst of the oxygen removal device 144 of the hydrocarbon production system 100. The oxygen removal catalyst of the oxygen removal device 144 according to this embodiment is, for example, a platinum-based catalyst, a palladium-based catalyst, or a nickel-based catalyst. The oxygen removal catalyst has, for example, a pellet shape or a honeycomb shape.

Further, in this embodiment, a hydrocarbon synthesis catalyst of the hydrocarbon production device 160 has higher tolerance to sulfur than that of the hydrocarbon synthesis catalyst of the hydrocarbon production device 160 of the hydrocarbon production system 100. The hydrocarbon synthesis catalyst of the hydrocarbon production device 160 according to this embodiment is, for example, a nickel-based catalyst or an iron-based catalyst. The hydrocarbon synthesis catalyst has, for example, a pellet shape or a honeycomb shape.

As described above, as compared to the hydrocarbon production system 100, the hydrocarbon production system 400 according to this embodiment allows the omission of the dry desulfurization device 142 and the catalytic desulfurization device 146. Thus, the hydrocarbon production system 400 enables the efficient use of thermal energy while reducing cost required for the dry desulfurization device 142 and the catalytic desulfurization device 146.

### Fifth Embodiment: Hydrocarbon Production System

In the first embodiment, there has been given, as an example, the case in which the hydrocarbon production system 100 extracts carbon dioxide from the mixed gas containing a sulfur component, oxygen, and carbon dioxide. However, when the mixed gas contains a sulfur component and carbon dioxide and scarcely contains oxygen, the dry desulfurization device 142 and the oxygen removal device 144 may be omitted.

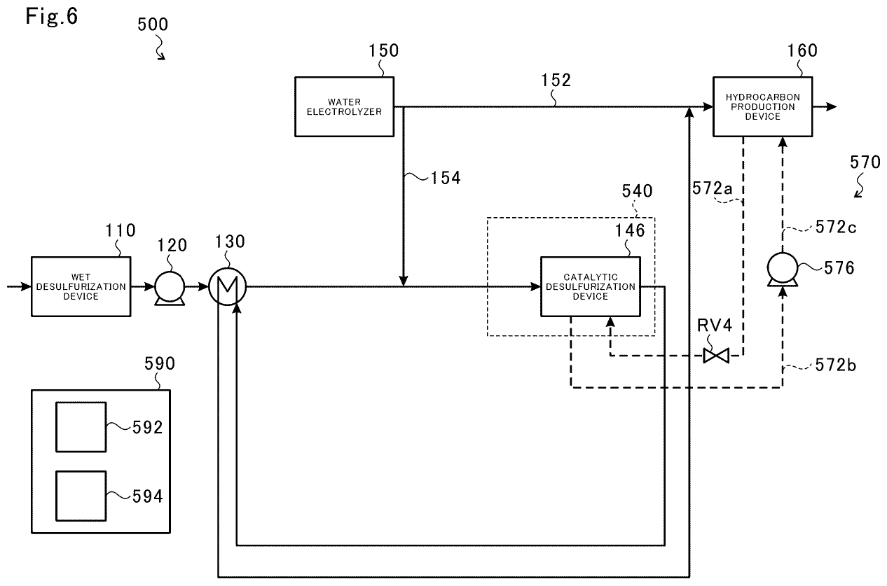

FIG. 6 is a diagram for illustrating a hydrocarbon production system 500 according to a fifth embodiment. As illustrated in FIG. 6, the hydrocarbon production system 500 includes a wet desulfurization device 110, a compressor 120, a heat exchanger 130, an impurity removal device 540, a water electrolyzer 150, a hydrocarbon production device 160, a heat supply unit 570, and a central controller 590. In FIG. 6, the solid-line arrows indicate the flows of gases such as a mixed gas, carbon dioxide, hydrogen, and hydrocarbon. Further, in FIG. 3, the broken-line arrows indicate the flows of heat media.

Substantially the same components as those in the above-mentioned hydrocarbon production system 100 are denoted by the same reference symbols, and description thereof is omitted.

In this embodiment, a mixed gas contains a sulfur component and carbon dioxide and scarcely contains oxygen. The mixed gas is, for example, an exhaust gas emitted from a coal-fired power plant.

The impurity removal device 540 includes a catalytic desulfurization device 146.

The heat supply unit 570 supplies reaction heat generated in the hydrocarbon production device 160 to the catalytic desulfurization device 146. In this embodiment, the heat supply unit 570 includes flow paths 572a to 572c and a feeding device 576.

The flow path 572a connects the hydrocarbon production device 160 and the catalytic desulfurization device 146 to each other. The flow path 572b connects the catalytic desulfurization device 146 and a suction side of the feeding device 576 to each other. The flow path 572c connects a discharge side of the feeding device 576 and the hydrocarbon production device 160 to each other.

The feeding device 576 is, for example, a pump. When the feeding device 576 is operated, a heat medium circulates through the hydrocarbon production device 160 and the catalytic desulfurization device 146 via the flow paths 572a to 572c. As a result, the reaction heat generated in the hydrocarbon production device 160 is supplied to the catalytic desulfurization device 146.

Further, a flow rate control valve RV4 is provided to the flow path 572a. An opening degree of the flow rate control valve RV4 is adjusted by a heat controller 592 described later.

The central controller 590 is formed of a semiconductor integrated circuit including a central processing unit (CPU). The central controller 590 reads out, for example, a program and a parameter each for operating the CPU from a ROM. The central controller 590 manages and controls the entire hydrocarbon production system 500 in cooperation with a RAM serving as a working area and other electronic circuit.

In this embodiment, the central controller 590 also functions as the heat controller 592 and an activation controller 594.

The heat controller 592 adjusts the opening degree of the flow rate control valve RV4 so that a temperature of the catalytic desulfurization device 146 is maintained at an activation temperature of the desulfurization catalyst. In this embodiment, the heat controller 592 adjusts the opening degree of the flow rate control valve RV4 so that the temperature of the catalytic desulfurization device 146 becomes 200° C. or higher and 300° C. or lower.

When the hydrocarbon production device 160 is to be activated, the activation controller 594 starts the operations of the compressor 120, the water electrolyzer 150, and the feeding device 576.

As described above, as compared to the hydrocarbon production system 100, the hydrocarbon production system 500 according to this embodiment allows the omission of the dry desulfurization device 142 and the oxygen removal device 144. Thus, the hydrocarbon production system 500 enables the efficient use of thermal energy while reducing cost required for the dry desulfurization device 142 and the oxygen removal device 144.

### Sixth Embodiment: Hydrocarbon Production System

In the first embodiment, there has been given, as an example, the case in which the hydrocarbon production system 100 extracts carbon dioxide from the mixed gas containing a sulfur component, oxygen, and carbon dioxide. However, when the mixed gas contains oxygen and carbon dioxide and scarcely contains a sulfur component, the wet desulfurization device 110, the dry desulfurization device 142, and the catalytic desulfurization device 146 may be omitted.

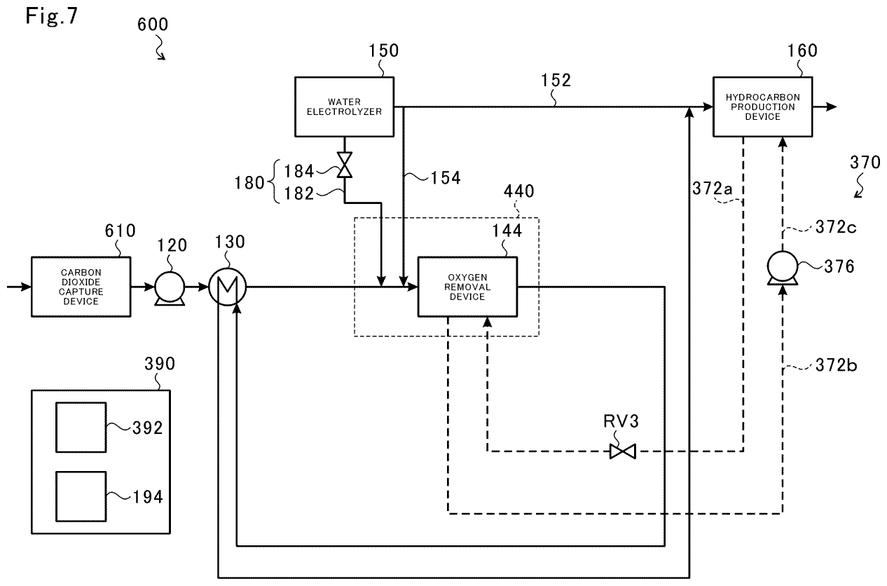

FIG. 7 is a diagram for illustrating a hydrocarbon production system 600 according to a sixth embodiment. As illustrated in FIG. 7, the hydrocarbon production system 600 includes a carbon dioxide capture device 610, a compressor 120, a heat exchanger 130, an impurity removal device 440, a water electrolyzer 150, a hydrocarbon production device 160, a heat supply unit 370, an activation device 180, and a central controller 390. In FIG. 7, the solid-line arrows indicate the flows of gases such as a mixed gas, carbon dioxide, oxygen, hydrogen, and hydrocarbon. Further, in FIG. 7, the broken-line arrows indicate the flows of heat media.

Substantially the same components as those in the above-mentioned hydrocarbon production systems 300 and 400 are denoted by the same reference symbols, and description thereof is omitted.

The carbon dioxide capture device 610 captures carbon dioxide from air. The carbon dioxide capture device 610 is, for example, a direct air capture (DAC) device. A mixed gas output from the carbon dioxide capture device 610 contains oxygen and carbon dioxide and scarcely contains a sulfur component.

In this embodiment, a suction side of the compressor 120 is connected to the carbon dioxide capture device 610. Further, a discharge side of the compressor 120 is connected to an oxygen removal device 144. Thus, the mixed gas output from the carbon dioxide capture device 610 is supplied to the oxygen removal device 144.

As described above, as compared to the hydrocarbon production system 100, the hydrocarbon production system 600 according to this embodiment allows the omission of the wet desulfurization device 110, the dry desulfurization device 142, and the catalytic desulfurization device 146. Thus, the hydrocarbon production system 600 enables the efficient use of thermal energy while reducing cost required for the wet desulfurization device 110, the dry desulfurization device 142, and the catalytic desulfurization device 146.

The embodiments have been described above with reference to the attached drawings, but, needless to say, the present disclosure is not limited to the embodiments described above. It is apparent that those skilled in the art may arrive at various alternations and modifications within the scope of claims, and those examples are construed as naturally falling within the technical scope of the present disclosure.

For example, in the first to sixth embodiments, there has been given, as an example, the case in which the heat supply unit 170, 370, 570 supplies the reaction heat generated in the hydrocarbon production device 160 to any one or both of the oxygen removal device 144 and the catalytic desulfurization device 146. However, the heat supply unit may supply the reaction heat generated in the hydrocarbon production device 160 to any one or more of the dry desulfurization device 142, the oxygen removal device 144, and the catalytic desulfurization device 146.

Further, in the first to sixth embodiments, there has been given, as an example, the case in which hydrogen produced by the water electrolyzer 150 is distributed to the impurity removal device 140, 240, 340, 440, 550, and the hydrocarbon production device 160. However, a full amount of hydrogen produced by the water electrolyzer 150 may be supplied to the hydrocarbon production device 160 after passing through the impurity removal device 140, 240, 340, 440, 550.

Further, in the first to sixth embodiments, there has been given, as an example, the case in which the hydrocarbon production system 100, 200, 300, 400, 500, 600 includes the water electrolyzer 150. However, the hydrocarbon production system may include any one or both of a hydrogen production device and a hydrogen cylinder in place of or in addition to the water electrolyzer 150. The hydrogen production device is, for example, a steam reforming device.

Further, in the first embodiment, there has been given, as an example, the case in which the heat media that have recovered the reaction heat generated in the hydrocarbon production device 160 first heat the catalytic desulfurization device 146 and then heat the oxygen removal device 144. However, the heat media that have recovered the reaction heat generated in the hydrocarbon production device 160 may first heat the oxygen removal device 144 and then heat the catalytic desulfurization device 146. Further, the heat media that have recovered the reaction heat generated in the hydrocarbon production device 160 may heat the oxygen removal device 144 and the catalytic desulfurization device 146 in parallel (at the same time).

Further, in the first to fourth and sixth embodiments, there has been given, as an example, the case in which the hydrocarbon production system 100, 200, 300, 400, 600 includes the activation device 180. However, the activation device 180 is not an indispensable constituent component.

Further, in the first to sixth embodiments, there has been given, as an example, the case in which the hydrocarbon production system 100, 200, 300, 400, 500, 600 includes the heat exchanger 130. However, the heat exchanger 130 is not an indispensable constituent component.

The present disclosure can contribute to, for example, Goal 7 “Ensure access to affordable, reliable, sustainable and modern energy for all” and Goal 13 “Take urgent action to combat climate change and its impacts” in Sustainable Development Goals (SDGs).

What is claimed is:

1. A hydrocarbon production system, comprising:
an impurity removal device that removes an impurity including any one or both of oxygen and a sulfur component from a mixed gas containing the impurity and carbon dioxide;
a hydrocarbon production device, which includes a hydrocarbon synthesis catalyst for promoting a reaction for synthesizing hydrocarbon from carbon dioxide and hydrogen and synthesizes the hydrocarbon from the carbon dioxide contained in the mixed gas having the impurity removed by the impurity removal device and hydrogen; and
a heat supply unit that supplies reaction heat generated in the hydrocarbon production device to the impurity removal device.

2. The hydrocarbon production system according to claim 1, further comprising a wet desulfurization device that removes the sulfur component from the mixed gas,
wherein the impurity removal device comprises a catalytic desulfurization device, which includes a desulfurization catalyst and is to be supplied with the mixed gas that has been treated by the wet desulfurization device, and
wherein the heat supply unit supplies the reaction heat to the catalytic desulfurization device.

3. The hydrocarbon production system according to claim 1, further comprising a wet desulfurization device that removes the sulfur component from the mixed gas,
wherein the impurity removal device comprises:
an oxygen removal device, which includes an oxygen removal catalyst and is to be supplied with the mixed gas that has been treated by the wet desulfurization device and hydrogen; and
a catalytic desulfurization device, which includes a desulfurization catalyst and is to be supplied with the mixed gas that has been treated by the oxygen removal device, and
wherein the heat supply unit supplies the reaction heat to any one or both of the catalytic desulfurization device and the oxygen removal device.

4. The hydrocarbon production system according to claim 1, further comprising a wet desulfurization device that removes the sulfur component from the mixed gas,
wherein the impurity removal device comprises:
a dry desulfurization device, which includes an adsorbent for adsorbing the sulfur component and is to be supplied with the mixed gas that has been treated by the wet desulfurization device;
an oxygen removal device, which includes an oxygen removal catalyst and is to be supplied with the mixed gas that has been treated by the dry desulfurization device and hydrogen; and
a catalytic desulfurization device, which includes a desulfurization catalyst and is to be supplied with the mixed gas that has been treated by the oxygen removal device, and
wherein the heat supply unit supplies the reaction heat to any one or more of the catalytic desulfurization device, the oxygen removal device, and the dry desulfurization device.

5. The hydrocarbon production system according to claim 1, further comprising a wet desulfurization device that removes the sulfur component from the mixed gas,
wherein the impurity removal device comprises an oxygen removal device, which includes an oxygen removal catalyst and is to be supplied with the mixed gas that has been treated by the wet desulfurization device and hydrogen, and
wherein the heat supply unit supplies the reaction heat to the oxygen removal device.

6. The hydrocarbon production system according to claim 1, further comprising a wet desulfurization device that removes the sulfur component from the mixed gas,
wherein the impurity removal device comprises:
a dry desulfurization device, which includes an adsorbent for adsorbing the sulfur component and is to be supplied with the mixed gas that has been treated by the wet desulfurization device; and
an oxygen removal device, which includes an oxygen removal catalyst and is to be supplied with the mixed gas that has been treated by the dry desulfurization device and hydrogen, and
wherein the heat supply unit supplies the reaction heat to any one or both of the oxygen removal device and the dry desulfurization device.

7. The hydrocarbon production system according to claim 1,
wherein the impurity removal device comprises an oxygen removal device, which includes an oxygen removal catalyst and is to be supplied with hydrogen, and
wherein the heat supply unit supplies the reaction heat to the oxygen removal device.

8. The hydrocarbon production system according to claim 3,
wherein the heat supply unit includes a heat medium for recovering the reaction heat generated in the hydrocarbon production device, and
wherein the heat medium heats the catalytic desulfurization device and then heats the oxygen removal device.

9. The hydrocarbon production system according to claim 2, further comprising a heat exchanger that allows heat exchange between the mixed gas that has been treated by the wet desulfurization device and the mixed gas that has been treated by the impurity removal device.

10. The hydrocarbon production system according to claim 3, further comprising:
a water electrolyzer that electrolyzes water to produce hydrogen and oxygen; and
an activation device that supplies, when the hydrocarbon production device is to be activated, the oxygen produced by the water electrolyzer,
wherein the hydrogen produced by the water electrolyzer is supplied to the oxygen removal catalyst and the hydrocarbon production device.

11. A hydrocarbon production method, comprising:
removing an impurity including any one or both of oxygen and a sulfur component from a mixed gas containing the impurity and carbon dioxide in an impurity removal device;
causing a reaction between the mixed gas having the impurity removed and hydrogen; and
supplying reaction heat generated in the reaction between carbon dioxide contained in the mixed gas having the impurity removed and the hydrogen to the impurity removal device.
